(19) United States
(12) Patent Application Publication
(10) Pub. No.: US 2025/0229274 A1
(43) Pub. Date: Jul. 17, 2025

(54) Multi-Use Sensor Holder

(71) Applicant: Merck Patent GmbH, Darmstadt (DE)

(72) Inventors: Joseph Geringer, Worcester, MA (US); Shawn Bates, Boston, MA (US); Christine Crequy, Molsheim (FR)

(21) Appl. No.: 18/853,691

(22) PCT Filed: Apr. 27, 2023
(86) PCT No.: PCT/US2023/020191

§ 371 Date: Oct. 2, 2024

Related U.S. Application Data
(60) Provisional application No. 63/335,963, filed on Apr. 28, 2022.

(51) Int. Cl. B01L 9/00; G01D 11/30

(52) U.S. Cl. CPC B01L 9/50; G01D 11/30; B01L 2200/021

(57) ABSTRACT

Probe holder for a bioreactor or other container or vessel, functioning as a base platform or stand to hold a sensor or probe, such as a Raman probe, in its an open position to be sterilized as part of an assembly including the sensor attached to a housing or container with an aseptic connector. The holder aids the user in positioning the sensor or probe at least partially within or in fluid communication with the interior of the bioreactor or vessel and holding and/or locking it in position during the use of the sensor or probe.

This application claims priority of U.S. Provisional Application Ser. No. 63/335,963 filed Apr. 28, 2022, the disclosure of which is hereby incorporated by reference.

### BACKGROUND

### Field of the Disclosure

This disclosure relates to the measurement of properties of fluids. More particularly, embodiments of the disclosure relate to holders and housings for probes and/or sensors for the measurement of fluids within a bioreactor, biocontainer or other vessel.

Cell cultures have been utilized for many years in life science and biopharmaceutical research and manufacturing. Cell culture systems depend on controlled environments for cell maintenance, growth, expansion, and testing. Despite often taking stringent measures to avoid outbreaks of contamination and the like, e.g., fungus or bacterial contamination, such outbreaks still occur, often with the impact of compromising weeks of research and halting operations for days or weeks. At the very least, results of cell culture assays can be distorted by unintended changes in cell physiology due to inconsistencies in the underlying cell culture. Researchers must be vigilant in monitoring and evaluating cell health by visually observing subtle changes in cell morphology, growth patterns, and growth rates that may signal problems with a particular culture.

Researchers that grow mammalian cells often find the maintenance of cell cultures to be a very time consuming task. Actions such as visually assessing the health of cells by determining cell morphology under a microscope, replacing media (feed), recovering the cells (confluency), dealing with contaminants, monitoring metabolites or cell interactions, and other issues must be carefully addressed.

Unfortunately, in the modern laboratory environment, there are many impediments to the proper monitoring of cultured cells. For example, it may be impractical and cost prohibitive to conduct around-the-clock (e.g., every 2-4 hours) manual examination of cell cultures. Additionally, manually monitoring cell cultures round-the-clock often takes a physical and mental toll on researchers, resulting in an overall diminished quality of life, and increasing the likelihood of an observational error due to excessive fatigue. Furthermore, fully automated cell culture monitoring systems are excessively cumbersome and complicated, and require the investment of large sums of money.

In addition, simple visual examination of cells provides only a subjective assessment, with no lasting visual record or archive. Signs of problems with cells and cell cultures can be missed, leading to serious deleterious impacts on the quality of data generated by cell-based assays. The ability to supply healthy living cell cultures is an ever growing problem in today's competitive multinational biological and biopharmaceutical industries.

A variety of sensors may be used with bioreactors, biocontainers, mixers and other vessels (e.g., disposable bags, etc.) that might need or benefit from measurements of various parameters inside the bioreactor mixer or vessel, such as dissolved oxygen content, pH, CO₂ content, glucose content, turbidity, viable cell density, etc. Measuring the physical properties of fluids in situ can present further disparities given small sample sizes traversing discrete, low-flow areas, which may not be representative of the larger population. The bioprocessing systems may contain conduits, typically part of a closed system, having fluid flow therethrough, and sampling through such conduits presents similar sampling problems.

In some bioprocessing, i.e., processing of monoclonal antibodies, capsids, cell lines, inline viral inactivation processes, and the like, measurements are taken from within a tank, e.g., a static measurement or from within fluid streams, e.g., several liters per minute, flowing through conduits. Furthermore, in some cases, the measurements can be performed on a sample taken from a finished product or the completion of a particular sub-operation. Sampling can be another vector for introducing undesirable contaminants into a process. All of the foregoing represents problems to overcome for processors trying to measure the properties of biological fluids.

More recently, trends in bioprocessing are biased toward providing continuous processing, i.e., intensified processing, wherein the biological fluid from one process is introduced to a separate process, which provides further challenges for sampling, measuring, and process monitoring. Process control is particularly challenging for intensified processing.

Process control in the demanding conditions of continuous processes requires the development of, at least, either new sensors or novel manners with which to incorporate existing probes and sensors, wherein the collection of reliable and accurate data is ensured while accommodating newer requirements, i.e., quick response times for inline flow continuous processes. Such measurements typically require large probes. Response times are a function of the kinematics of the chemical/biological processes, which are typically slower than mechanical ones. This explains the complexity of, for example, conductivity or pH measurements as compared with, for example, sensing the pressure of a fluid.

Recent technologies have attempted, with existing probes and sensors for the measurement of pH, viable cell density (VCD), conductivity, turbidity, dissolved oxygen, and temperature, to optimize and improve their accuracy, reliability and stability over long process trials. The placement of sensors within a process is also a significant factor.

A probe holder that operates as a platform to hold a sensor in an open position for sterilization, e.g., via autoclave, gamma or ethylene oxide (ETO), as part of an assembly comprising a sensor attached to a housing having a connector, such as an aseptic connector; operates as a holder to semi-permanently attach to a bioreactor, biocontainer, or vessel that aids a user to make a sterile connection and that aids a user in placing and holding a sensor into position within the bioreactor, biocontainer, or vessel during the use of the sensor represents an advance in the art.

It would be desirable to provide such a holder that serves one or more functions in order to help satisfy this benefit.

### SUMMARY

Problems of the prior art have been addressed by embodiments disclosed herein which include a sensor or probe holder for a bioreactor or other container or vessel. In some embodiments, a probe holder for positioning and holding a probe for engagement with a container is disclosed, the probe having a longitudinal axis and being extendable in a longitudinal direction between a compressed position and an extended position, the holder comprising a first translatable member and a second translatable member, the first and second translatable members being translatable with respect to each other and configured to support said probe in said extended position and engage said probe in said compressed position. In some embodiments, the holder functions as a base platform or stand to hold a sensor or probe in an open position to be sterilized such as via autoclave, gamma or ETO as part of an assembly including the sensor attached to a housing or container with an aseptic connector. In some embodiments, the holder serves as a holder to position and semi-permanently attach the sensor or probe to a bioreactor, mixer or vessel aiding the user to make sterile connection easier. In some embodiments, the holder aids the user in positioning the sensor or probe at least partially within or in fluid communication with the interior of the bioreactor or vessel and holding and/or locking it in position during the use of the sensor or probe. In some embodiments, a portion of the sensor or probe physically contacts the contents of the bioreactor, mixer or vessel when properly positioned by the holder, enabling the sensor or probe to measure or analyze one or more parameters of the contents in an efficient, reliable and repeatable manner. In some embodiments, the holder permanently or semi-permanently (e.g., removably) fixes the sensor or probe to the bioreactor, container or vessel. In some embodiments, the probe is a Raman probe.

In certain embodiments, the probe holder is designed to perform inline and in situ measurements. Embodiments described herein include a holder that supports and holds a sensor or probe in place as an entire assembly is autoclaved or otherwise sterilized. The holder allows a user to place the assembly in position before aligning and engaging connectors, for example, sterile connectors. The holder locks and holds a sensor or probe in position during processing and/or measuring of a sample in a container such as a bioreactor. The holder may be shipped assembled with a bellows and a sterile connector, wherein a user mates a chosen sensor therewith. The bellows, if present, can be expandable and collapsible and protects the sensor from damage, etc., by surrounding the sensor or probe. After assembling the sensor or probe to the holder, the assembly may be sterilized. After sterilization, the user coordinates the holder assembly with the bioreactor/biocontainer/vessel. The user may then connect a sterile connector and, for e.g., optionally remove a sterile barrier tab. After the sterile connector is opened, fluid can be introduced into the bioreactor/biocontainer/vessel and parameters thereof measured with the sensor or probe.

In some embodiments, the first and second translatable members are linearly translatable in the direction of the longitudinal axis.

In some embodiments, the first and second translatable members are pivotably translatable about a pivot axis orthogonal to the longitudinal axis.

In some embodiments, the probe holder comprises an expandable and collapsible bellows having a passageway configured to receive at least a portion of the probe.

In some embodiments, the first translatable member protects the expandable and collapsible bellows when the expandable and collapsible bellows is in a collapsed state.

In some embodiments, the holder further comprises an aseptic or sterile connector.

In some embodiments, the first translatable member slides in a slot or groove of the second translatable member.

In some embodiments, disclosed is a probe holder for positioning and holding a probe for engagement with a container, the probe having a longitudinal axis and being extendable in a longitudinal direction between a compressed position and an extended position, the holder comprising a slotted base, a translatable member and a support member, the translatable member being translatable in the slotted base with respect to the support member. In some embodiments, the translatable member comprises a movable block that is lockable in the base. In some embodiments, the translatable member is U-shaped. In some embodiments, the probe holder includes a clamp assembly having a U-shaped recess and a locking bar that is positionable to traverse the U-shaped recess. In some embodiments, the clamp assembly has a first free end and a second free end defines therebetween the U-shaped recess, and the locking bar is pivotable on the first free end. In some embodiments, the slotted base has two opposite spaced elongated side walls defining a channel between them.

### BRIEF DESCRIPTION OF THE DRAWINGS

The embodiments disclosed herein may take form in various components and arrangements of components, and in various process operations and arrangements of process operations. The drawings are only for purposes of illustrating preferred embodiments and are not to be construed as limiting. This disclosure includes the following drawings.

FIG. 1A is a perspective view of a holder shown in a first position in accordance with a first embodiment;

FIG. 1B is a perspective view of a holder shown in a second position in accordance with a first embodiment;

FIG. 1C is a side view of a holder shown in a first position in accordance with a first embodiment;

FIG. 1D is a side view of a holder shown in a second position in accordance with a first embodiment;

FIG. 1E is a front view of a holder shown in a first position in accordance with a first embodiment;

FIG. 1F is a front view of a holder shown in a second position in accordance with a first embodiment;

FIG. 1G is a rear view of a holder shown in a first position in accordance with a first embodiment;

FIG. 1H is a rear view of a holder shown in a second position in accordance with a first embodiment;

FIG. 1I is a top view of a holder shown in a first position in accordance with a first embodiment;

FIG. 1J is a bottom view of a holder shown in a first position in accordance with a first embodiment;

FIG. 2A is a perspective view of a holder shown in a first position in accordance with a first embodiment, including a generic coupling device;

FIG. 2B is a perspective view of a holder shown in a second position in accordance with a first embodiment, including a generic coupling device;

FIG. 3A is a top view of a container showing the holder of FIG. 1 coupled to a port thereof and another holder of FIG. 1 positioned to be coupled to another port thereof in accordance with the first embodiment;

FIG. 3B is a first perspective view of the container of FIG. 3A;

FIG. 3C is a second perspective view of the container of FIG. 3A;

FIG. 4A is a perspective view of a holder shown in a first position in accordance with a second embodiment;

FIG. 4B is a perspective view of a holder shown in a second position in accordance with a second embodiment;

FIG. 4C is a side view of a holder shown in a first position in accordance with the second embodiment;

FIG. 4D is a side view of a holder shown in a second position in accordance with the second embodiment;

FIG. 4E is a front view of a holder shown in a first position in accordance with the second embodiment;

FIG. 4F is a front view of a holder shown in a second position in accordance with the second embodiment;

FIG. 4G is a rear view of a holder shown in a first position in accordance with the second embodiment;

FIG. 4H is a rear view of a holder shown in a second position in accordance with the second embodiment;

FIG. 4I is a side view of a holder shown in an intermediate position in accordance with the second embodiment;

FIG. 5A is a perspective view of a holder shown in a first position in accordance with the second embodiment, including a generic coupling device;

FIG. 5B is a perspective view of a holder shown in a second position in accordance with the second embodiment, including a generic coupling device;

FIG. 6A is a perspective view of a holder shown in a first position in accordance with a third embodiment;

FIG. 6B is a perspective view of a holder shown in a second position in accordance with a third embodiment;

FIG. 6C is a side view of a holder shown in a first position in accordance with the third embodiment;

FIG. 6D is a side view of a holder shown in a second position in accordance with the third embodiment;

FIG. 6E is a front view of a holder shown in a first position in accordance with the third embodiment;

FIG. 6F is a front view of a holder shown in a second position in accordance with the third embodiment;

FIG. 6G is a rear view of a holder shown in a first position in accordance with the third embodiment;

FIG. 6H is a rear view of a holder shown in a second position in accordance with the third embodiment;

FIG. 6I is a bottom view of a holder shown in a second position in accordance with the third embodiment;

FIG. 6J is a top view of a holder shown in a second position in accordance with the third embodiment;

FIG. 7 is a perspective view showing the holder of FIG. 6B coupled to a port thereof and another holder of FIG. 6B positioned to be coupled to another port thereof in accordance with the third embodiment;

FIG. 8A is a perspective view of a holder shown in a first position in accordance with a fourth embodiment;

FIG. 8B is a perspective view of a holder shown in a second position in accordance with a fourth embodiment;

FIG. 8C is a side view of a holder shown in a first position in accordance with the fourth embodiment;

FIG. 8D is a side view of a holder shown in a second position in accordance with the fourth embodiment;

FIG. 8E is a front view of a holder shown in a first position in accordance with the fourth embodiment;

FIG. 8F is a front view of a holder shown in a second position in accordance with the fourth embodiment;

FIG. 8G is a rear view of a holder shown in a first position in accordance with the fourth embodiment;

FIG. 8H is a rear view of a holder shown in a second position in accordance with the fourth embodiment;

FIG. 8I is a top view of a holder shown in a second position in accordance with the fourth embodiment;

FIG. 9 is a perspective view showing the holder of FIG. 8B coupled to a port thereof and another holder of FIG. 8A positioned to be coupled to another port thereof in accordance with the fourth embodiment;

FIG. 10A is perspective view of a holder and sensor shown in a first position in accordance with a fifth embodiment;

FIG. 10B is a top view of the holder and sensor of FIG. 10A;

FIG. 10C is a side view of the holder and sensor of FIG. 10A;

FIG. 10D is a bottom view of the holder and sensor of FIG. 10A;

FIG. 10E is a front view of the holder and sensor of FIG. 10A;

FIG. 10F is a rear view of the holder and sensor of FIG. 10A;

FIG. 11A is perspective view of the holder of FIG. 10A shown without a sensor;

FIG. 11B is a top view of the holder of FIG. 10A shown without a sensor;

FIG. 11C is a side view of the holder of FIG. 10A shown without a sensor;

FIG. 11D is a bottom view of the holder of FIG. 10A shown without a sensor;

FIG. 11E is a front view of the holder of FIG. 10A shown without a sensor;

FIG. 11F is a rear view of the holder of FIG. 10A shown without a sensor;

FIG. 12A is perspective view of a holder and sensor shown in a second position in accordance with the fifth embodiment;

FIG. 12B is a top view of the holder and sensor of FIG. 12A;

FIG. 12C is a side view of the holder and sensor of FIG. 12A;

FIG. 12D is a bottom view of the holder and sensor of FIG. 12A;

FIG. 12E is a front view of the holder and sensor of FIG. 12A;

FIG. 12F is a rear view of the holder and sensor of FIG. 12A;

FIG. 12G is another perspective view of a holder and sensor shown in the second position in accordance with the fifth embodiment;

FIG. 12H is another perspective view of holder and sensor shown in a first position in accordance with the fifth embodiment;

FIG. 13A is perspective view of a holder and sensor shown in a first position in accordance with a sixth embodiment;

FIG. 13B is a top view of the holder and sensor of FIG. 13A;

FIG. 13C is a side view of the holder and sensor of FIG. 13A;

FIG. 13D is a bottom view of the holder and sensor of FIG. 13A;

FIG. 13E is a front view of the holder and sensor of FIG. 13A;

FIG. 13F is a rear view of the holder and sensor of FIG. 13A;

FIG. 14A is perspective view of the holder of FIG. 13A shown without a sensor;

FIG. 14B is a top view of the holder of FIG. 13A shown without a sensor;

FIG. 14C is a side view of the holder of FIG. 13A shown without a sensor;

FIG. 14D is a bottom view of the holder of FIG. 13A shown without a sensor;

FIG. 14E is a front view of the holder of FIG. 13A shown without a sensor;

FIG. 14F is a rear view of the holder of FIG. 13A shown without a sensor;

FIG. 15A is perspective view of a holder and sensor shown in a second position in accordance with the sixth embodiment;

FIG. 15B is a top view of the holder and sensor of FIG. 15A;

FIG. 15C is a side view of the holder and sensor of FIG. 15A;

FIG. 15D is a bottom view of the holder and sensor of FIG. 15A;

FIG. 15E is a front view of the holder and sensor of FIG. 15A;

FIG. 15F is a rear view of the holder and sensor of FIG. 15A;

FIG. 16A is perspective view of a holder and sensor shown in a first position in accordance with a seventh embodiment;

FIG. 16B is a top view of the holder and sensor of FIG. 16A;

FIG. 16C is a side view of the holder and sensor of FIG. 16A;

FIG. 16D is a bottom view of the holder and sensor of FIG. 16A;

FIG. 16E is a front view of the holder and sensor of FIG. 16A;

FIG. 16F is a rear view of the holder and sensor of FIG. 16A;

FIG. 17A is perspective view of the holder of FIG. 16A shown without a sensor;

FIG. 17B is a top view of the holder of FIG. 16A shown without a sensor;

FIG. 17C is a side view of the holder of FIG. 16A shown without a sensor;

FIG. 17D is a bottom view of the holder of FIG. 16A shown without a sensor;

FIG. 17E is a front view of the holder of FIG. 16A shown without a sensor;

FIG. 17F is a rear view of the holder of FIG. 16A shown without a sensor;

FIG. 18A is perspective view of a holder and sensor shown in a second position in accordance with the sixth embodiment;

FIG. 18B is a top view of the holder and sensor of FIG. 18A;

FIG. 18C is a side view of the holder and sensor of FIG. 18A;

FIG. 18D is a bottom view of the holder and sensor of FIG. 18A;

FIG. 18E is a front view of the holder and sensor of FIG. 18A; and

FIG. 18F is a rear view of the holder and sensor of FIG. 18A.

### DETAILED DESCRIPTION

A more complete understanding of the components, processes and apparatuses disclosed herein can be obtained by reference to the accompanying drawing. The figures are merely schematic representations based on convenience and the ease of demonstrating the present disclosure, and is, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawing, and are not intended to define or limit the scope of the disclosure. In the drawing and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2 inches to 10 inches” is inclusive of the endpoints, 2 inches and 10 inches, and all the intermediate values).

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that many of the terms used herein are relative terms. For example, the terms “upper” and “lower” are relative to each other in location, i.e. an upper component is located at a higher elevation than a lower component, and should not be construed as requiring a particular orientation or location of the structure. As a further example, the terms “interior”, “exterior”, “inward”, and “outward” are relative to a center, and should not be construed as requiring a particular orientation or location of the structure.

The terms “top” and “bottom” are relative to an absolute reference, i.e. the surface of the earth. Put another way, a top location is always located at a higher elevation than a bottom location, toward the surface of the earth.

The terms “horizontal” and “vertical” are used to indicate direction relative to an absolute reference, i.e. ground level. However, these terms should not be construed to require structures to be absolutely parallel or absolutely perpendicular to each other.

The terms “sensor” and “probe” are used interchangeably herein and refer to any measurement or other device suitable for the application.

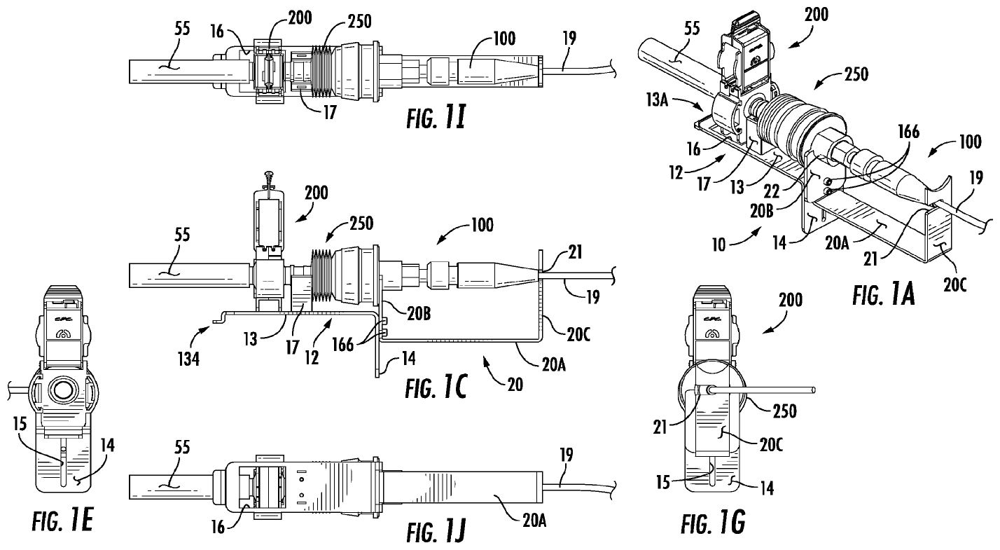

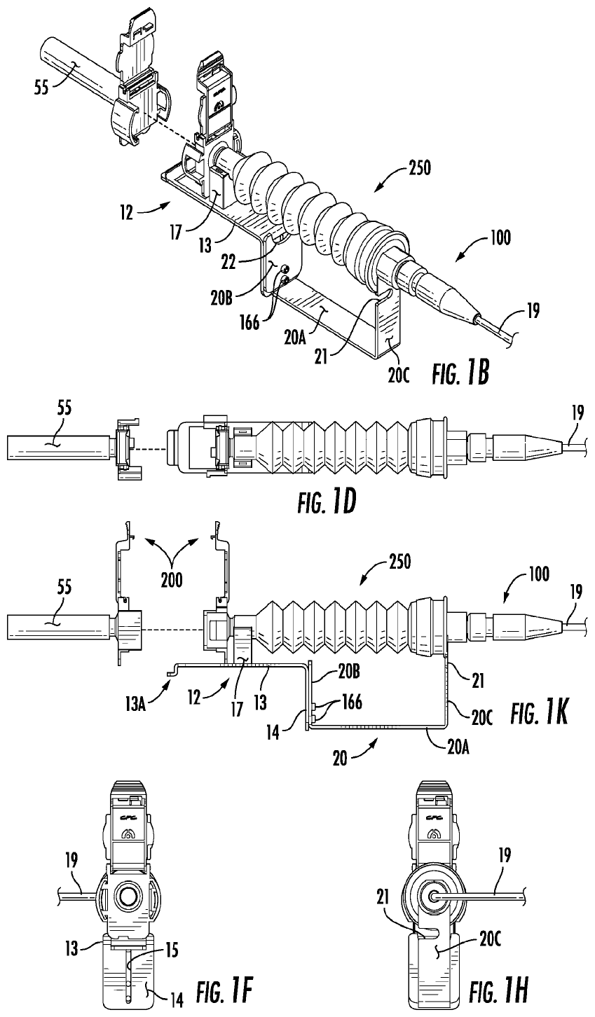

Turning now to FIGS. 1A through 1J, there is shown a first embodiment of a holder 10. FIGS. 1A, 1C, 1E, 1G, 1I and 1J depict the holder 10 supporting a sensor 100 just prior to insertion of the sensor into a container or vessel. In this position, the sensor 100 is supported and engaged by the holder 10. FIGS. 1B, 1D, 1F and 1H show the holder 10 supporting the sensor 100 such as for sterilization, e.g., autoclaving, where the sensor 100 is shown in an extended position, and is not engaged by the holder 10 (e.g., cord 19 of the sensor 100 is not inserted in radial groove 21), and the connector 200, e.g., a sterile or aseptic connector, is not in a connected position. In some embodiments, the holder 10 includes an expandable and collapsible bellows 250 having an internal passageway configured to receive a portion of the probe 100. In some embodiments, the collapsible bellows 250 is part of an assembly that includes a connector 200 at one end and a probe adapter on the other end. This assembly may be wired to a support block as part of the holder assembly on the connector end and supported on the opposite probe end.

In the embodiment shown in FIGS. 1A and 1B, the holder 10 includes an L-shaped slotted member 12 having an elongated support leg 13 (which may include an aperture 16 as a design detail to allow for clearance for the connector) and a slotted leg 14. The slotted leg 14 is at one end of the support leg 13, extends orthogonally thereto, and is spaced from an opposite free end 13a of the support leg 13. The slot 15 in the slotted leg 14 optionally bisects the slotted leg the longitudinal direction (vertically as depicted in FIGS. 1E and 1G). A probe support block 17 sits on the slotted leg 13 to aid in supporting the probe 100. The holder 10 of FIGS. 1A and 1B also includes a U-shaped member 20, with a base 20A, and respective first and second spaced orthogonally extending legs 20B and 20C. Leg 20B may include a groove or U-shaped region 22 at its free end shaped and positioned to receive and support a region of the sensor 100 as shown in FIG. 1A and 1G, such as an arcuate groove. Leg 20C may include a radial groove 21 shaped and positioned to receive and support a region of the probe 100 as shown in FIG. 1B. Leg 20B also includes one or more prongs 166 (two shown in FIGS. 1A and 1C) that are received by, engage with and travel in slot 15 of slotted leg 14. Thus the slot 15 is configured to receive the prong or prongs 166 or the like for sliding engagement. The length of the slot 15 determines the extent to which the slotted leg 14 may be raised or lowered with respect to leg 20B. Upon raising or lowering the slotted leg 14 to its desired position, the prong or prongs 166, which can be fasteners such as screws, can be tightened to secure the slotted leg 14 in that position. Accordingly, to engage and support the sensor 100, the leg 20B may be raised and fixed in position, whereby the groove 22 supports a region of the sensor 100 and the radial groove 21 receives a portion of the sensor 100 as shown in FIG. 1A.

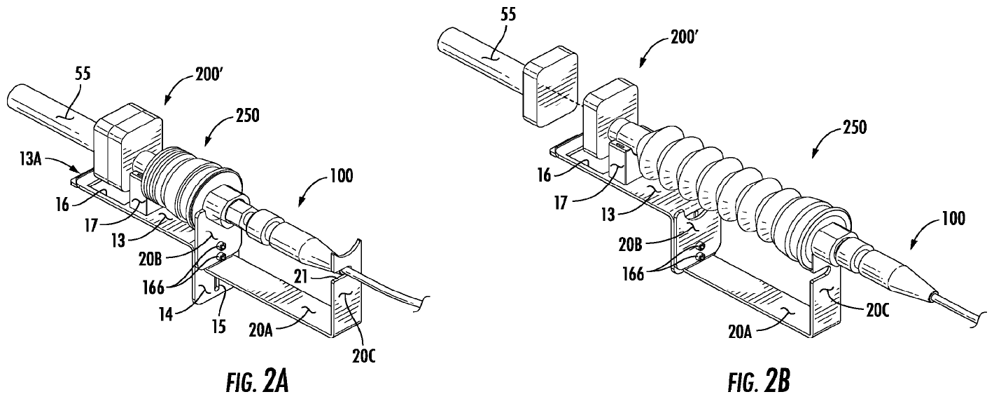

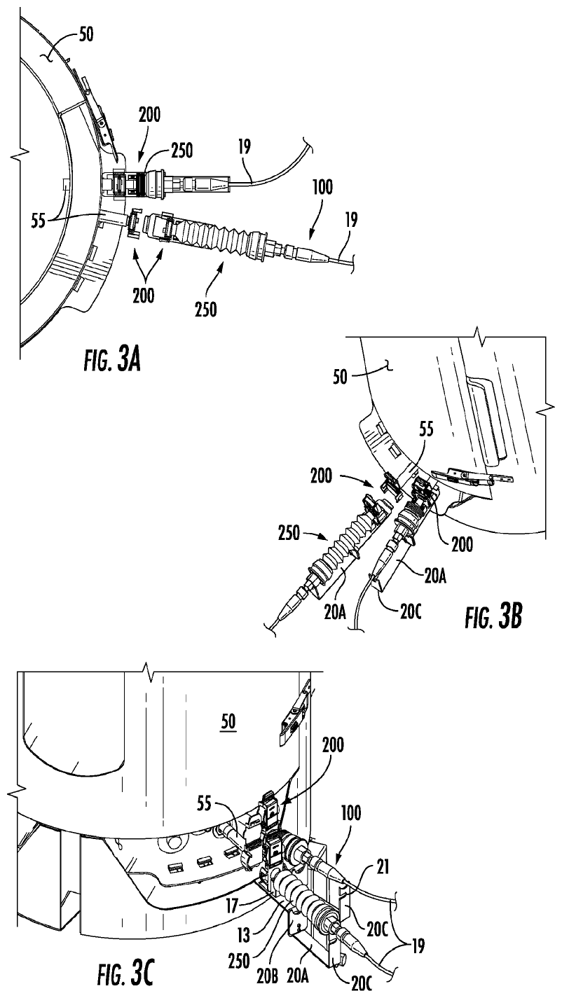

The sensor 100 may be connected to a port of a container 50 via a conduit 55 or the like with a suitable coupling device 200, such as an AseptiQuik® G connector commercially available from Colder Products Company. Other coupling devices may be used, as illustrated by the generic coupling device 200′ shown in FIGS. 2A (sensor in compressed position) and 2B (sensor 100 in expanded position), suitably accommodated by the holder 10. The connection to the port of the container 50 may be made via a conduit 55 or the like (FIGS. 3A, 3B, 3C) in fluid communication with the container interior. FIGS. 3A, 3B and 3C show a first sensor and holder assembly attached to a first conduit 55 in fluid communication with an interior volume of container 50 via a coupling 200, such as an AseptiQuik® G connector commercially available from Colder Products Company, such as disclosed in U.S. Pat. Nos. 7,631,660 and 10,871,250, the disclosures of which are hereby incorporated by reference, and a second sensor and holder assembly about to be attached to a second conduit 55 in fluid communication with an interior volume of container 50 via a coupling 200, such as an AseptiQuik® G connector. Other suitable connectors may be used.

In certain embodiments, in its supporting position shown in FIGS. 1B, 1D, 1F and 1H, the holder 10 serves to support the probe 100 during sterilization, such as during autoclaving. In this embodiment, the probe 100 is inserted through the bellows 250 and rests on block 17 and leg 20C, and the leg 20B is located out of the way of (e.g., below) the bellows 250 so as not to interfere with its expansion. To deploy the probe 100, the holder 10 is actuated so that support leg 13 is moved downwardly relative to base 20A (or base 20A is moved upwardly with respect to support leg 13), with prongs 166 sliding in slot 15, until the deploying position of FIGS. 1A, 1C, 1E and 1G is achieved. In this position, the probe 100 is held in arcuate recess 22 and groove 21 as shown, is also supported by block 17, and the bellows 250 is in a compressed state as shown. The assembly then may be coupled to a bioreactor 50 or other container, by positioning it for such coupling as exemplified in FIGS. 3A, 3B and 3C.

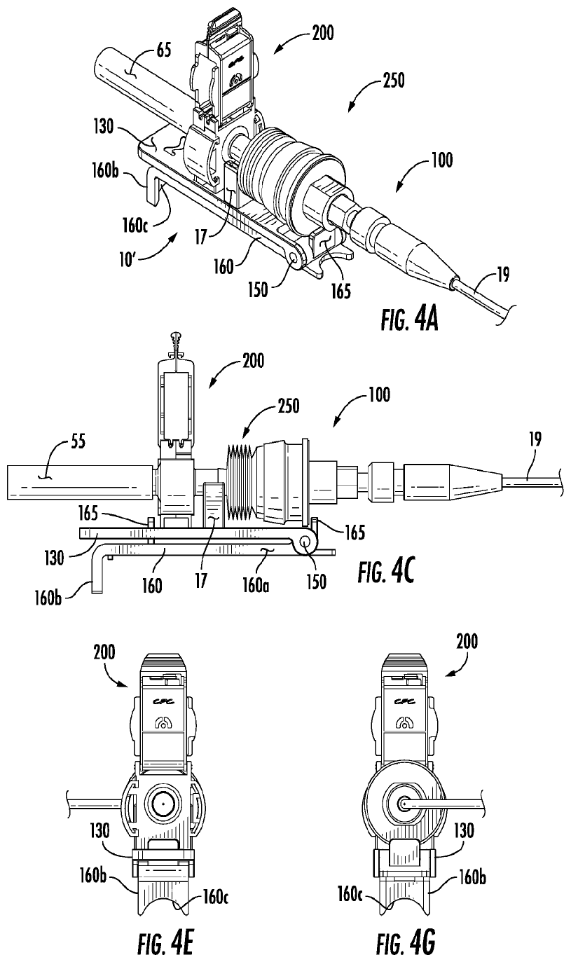

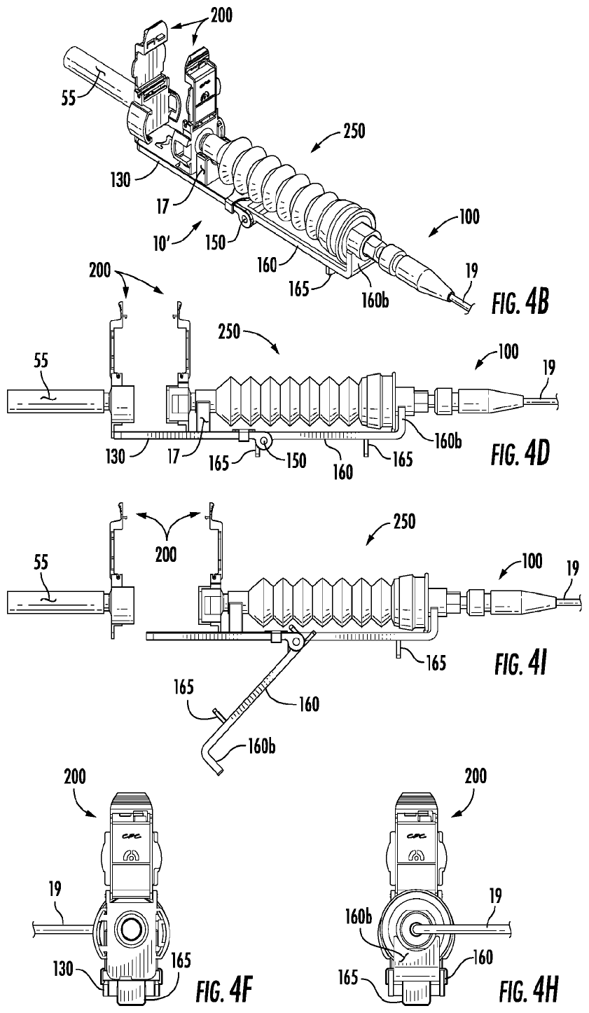

FIGS. 4A through 4H illustrate a second embodiment of a holder 10′ for a probe or sensor 100. FIG. 4A depicts the holder 10′ supporting a probe 100 just prior to insertion of the probe into a container or vessel such as a bioreactor, with bellows 250 in a compressed state. In this position, the probe 100 is supported and engaged by the holder 10′. As in other embodiments, the collapsible bellows 250 is part of an assembly that includes an aseptic connector one end and a probe adapter on the other end. This assembly may be wired to a support block as part of the holder assembly on the connector end and supported on the opposite probe end. FIG. 4B shows the holder 10′ supporting the probe 100 in a flat orientation such as for sterilization, e.g., autoclaving, where the probe 100 (and bellows 250) is shown in an extended position, and is not engaged by the holder 10′. Probe supporting block 17 and leg 160b of arm 160 function as probe supports as discussed below.

In this embodiment, the holder 10′ includes respective first and second arms 130, 160 pivotally connected together about a pin 150 or the like defining a pivot axis. In the embodiment shown in FIGS. 4A and 4B, the second arm 160 is L-shaped, having an elongated main portion 160a terminating in a generally orthogonally extending leg 160b. The leg 160b has an arcuate free end 160c as best seen in FIGS. 4E and 4G, configured to support the probe 100 when in the position of FIG. 4B. In the position of FIG. 4A, the leg 160b acts as a foot or stand and may support the probe 100.

To actuate the holder 10′ from its probe support position of FIG. 4B to its container probe engaging position of FIG. 4A, the second arm 160 is pivoted relative to first arm 130 about pin 150 (FIG. 4I), such as 180° or about 180°, causing leg 160b to pivot from an oriented upward position shown in FIG. 4B to an oriented downward position, underneath first arm 130, as shown in FIG. 4A. The bellows 250 is then compressed. In the position of FIG. 4A, the leg 160b may be supported on a substrate.

In some embodiments, the second arm 160 includes one or more feet 165 (two shown) extending in a direction opposite orthogonally extending leg 160b, as best seen in FIGS. 4C, 4D, 4F and 4G. When the holder 10′ is in the position shown in FIG. 4B, the one or more feet 165 function to support the holder (and probe 100) on a substrate, for engagement of the probe 100 with a container such as a bioreactor.

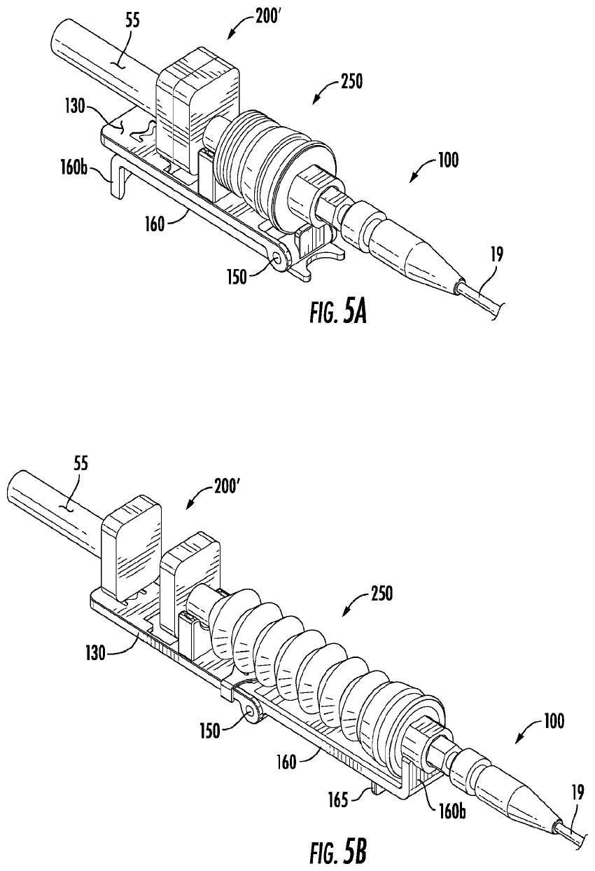

The sensor 100 may be connected to a port of a container 50 via a conduit 55 or the like with a suitable coupling device 200, such as an AseptiQuik® G. Other coupling devices may be used, as illustrated by the generic coupling device 200′ shown in FIGS. 5A (sensor in compressed position) and 5B (sensor in expanded position).

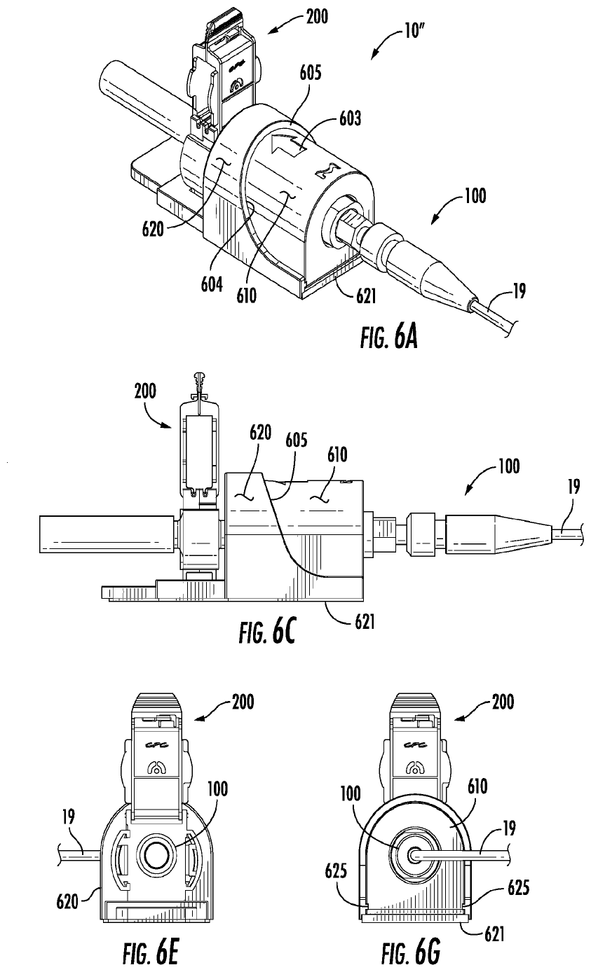

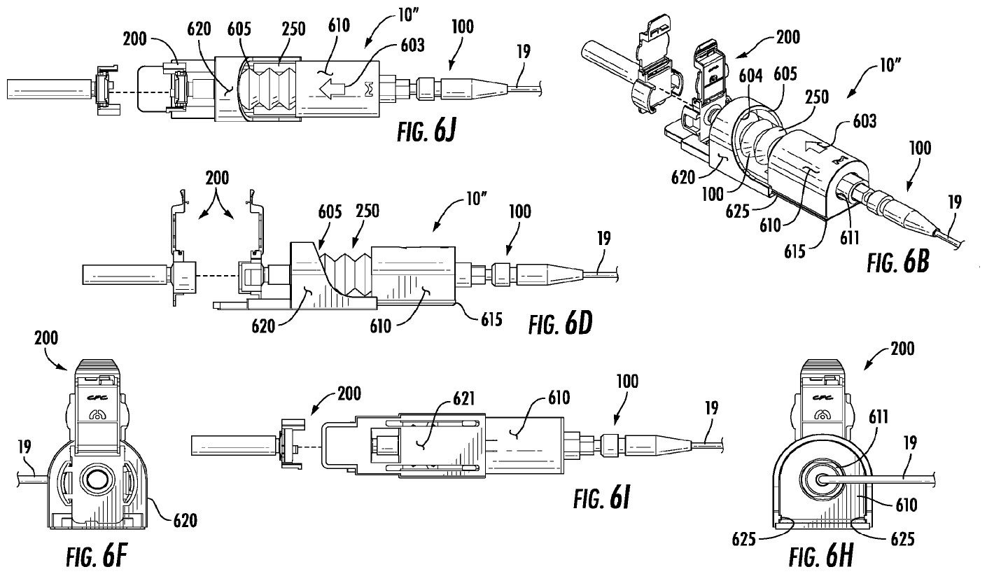

FIG. 6A illustrates a third embodiment of a sensor holder 10″. In its supporting position shown in FIGS. 6B, 6D, 6F, 6H, 6I and 6J, the holder 10″ serves to support the probe 100 during sterilization, such as during autoclaving. In this position, the bellows 250 is extended and exposed, as best seen in FIGS. 6B and 6D. As in other embodiments, the collapsible bellows 250 is part of an assembly that includes an aseptic connector one end and a probe adapter on the other end. This assembly may be wired to a support block as part of the holder assembly on the connector end and supported on the opposite probe end. The holder 10″ includes a main body 610 and a sleeve 620. The main body 610 and sleeve 620 are slidable with respect to each other, such as by linear translation, between a first position shown in FIG. 6B and a second position shown in FIG. 6A. In some embodiments, the sleeve 620 has a curvilinear profile 605, a bore 604 and a base 621 (FIG. 6I) that is wider than the main body 610, allowing the main body to translate linearly with respect to the arm 610 and thus slide into and out of the sleeve 620. For example, the main body 610 may include an elongated groove or slot 615 on opposite sides of the main body, positioned and dimensioned to receive a corresponding tongue, rib or protrusion 625 on opposite sides of the sleeve 620 (e.g., a tongue and groove structure). The ribs 625 are slidable in respective slots 615. Alternatively, the main body 610 could have the ribs and the sleeve 620 the slots. The main body 610 has a through hole or bore 611 configured to receive a portion of the probe 100 as shown.

To deploy the probe 100, once the probe 100 is situated in the holder 10″, the holder 10″ is actuated by sliding the main body 610 relative to the sleeve 620 (e.g., in the direction of arrow 603) so that the sleeve 610 receives a free end of the main body 610 as shown in FIGS. 6A and 6C, covering or surrounding and protecting the now compressed bellows 250. The probe 100 may be connected to a port of a container 50 via a conduit 55 or the like with a suitable coupling device 200, such as an AseptiQuik® G connector commercially available from Colder Products Company. Other coupling devices may be used, as illustrated by the generic coupling device 200′ shown in FIG. 7.

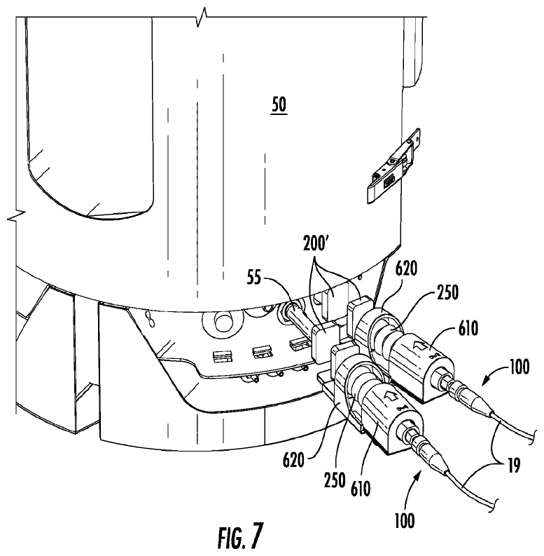

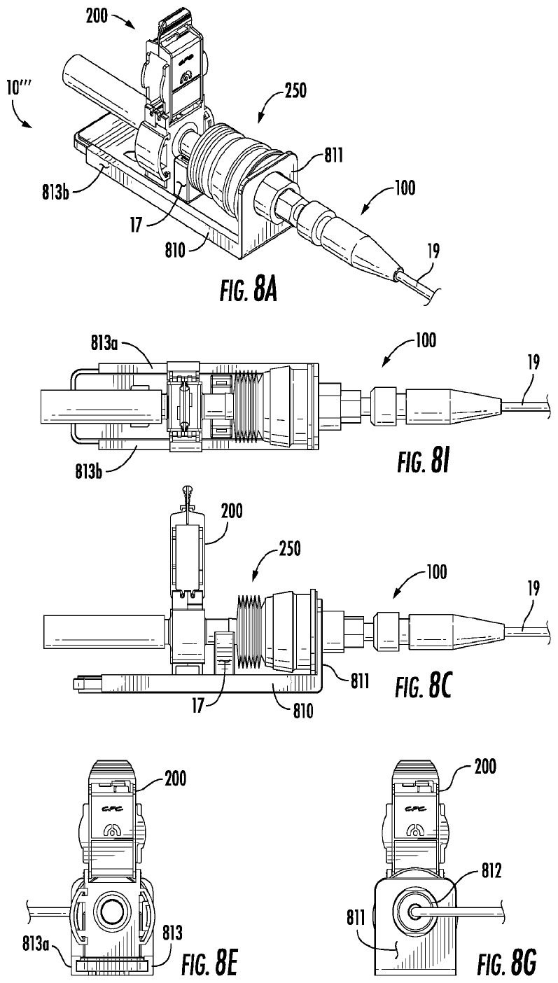

FIG. 8A illustrates a fourth embodiment of a sensor holder 10″′. In its supporting position shown in FIGS. 8B, 8D, 8F and 8H, the holder 10″′ serves to support the probe 100 during sterilization, such as during autoclaving. In this position, the bellows 250 is extended as best seen in FIGS. 8B and 8D. As in other embodiments, the collapsible bellows 250 is part of an assembly that includes an aseptic connector one end and a probe adapter on the other end. This assembly may be wired to a support block as part of the holder assembly on the connector end and supported on the opposite probe end. The holder 10″′ includes a main body 810 and an arm 820. The main body 810 is generally L-shaped, with an orthogonally extending arm 811 at one end thereof, the arm 811 including a through hole 812 for receiving the sensor 100. Side edges 813a, 813b (FIG. 81) of main body 810 are C-shaped (FIG. 8E), each defining a respective side groove for sliding engagement of arm 820. Thus the main body 810 and arm 820 are slidable with respect to each other, between a first position shown in FIG. 8B and a second position shown in FIG. 8A. In certain embodiments, the arm 820 translates lineally in the side grooves of the main body 810.

To deploy the probe 100, once the probe 100 is situated in the holder 10′″, the holder 10′″ is actuated by sliding the arm 820 relative to the main body 810, compressing the holder 10′″ (and the bellows 250) as shown in FIGS. 8A and 8C. The probe 100 may be connected to a port of a container 50 via a conduit 55 or the like with a suitable coupling device 200, such as an AseptiQuik® G connector commercially available from Colder Products Company. Other coupling devices may be used, as illustrated by the generic coupling device 200′ shown in FIG. 9.

In some embodiments, the holder is shipped assembled with the bellows and sterile connector and the user threads in their choice of sensor. After assembling the sensor to the holder, the assembly may be sterilized such as by autoclaving. After sterilization, the user may assemble the holder assembly to the bioreactor/vessel 50 via an alignment cleat, freeing up the user's hands to connect the sterile connector and pull the sterile barrier tab associated with the sterile connector. After the sterile connector is open, a clamp can be released from the tube and the sensor 100 can be pushed into the fluid in the vessel 50.

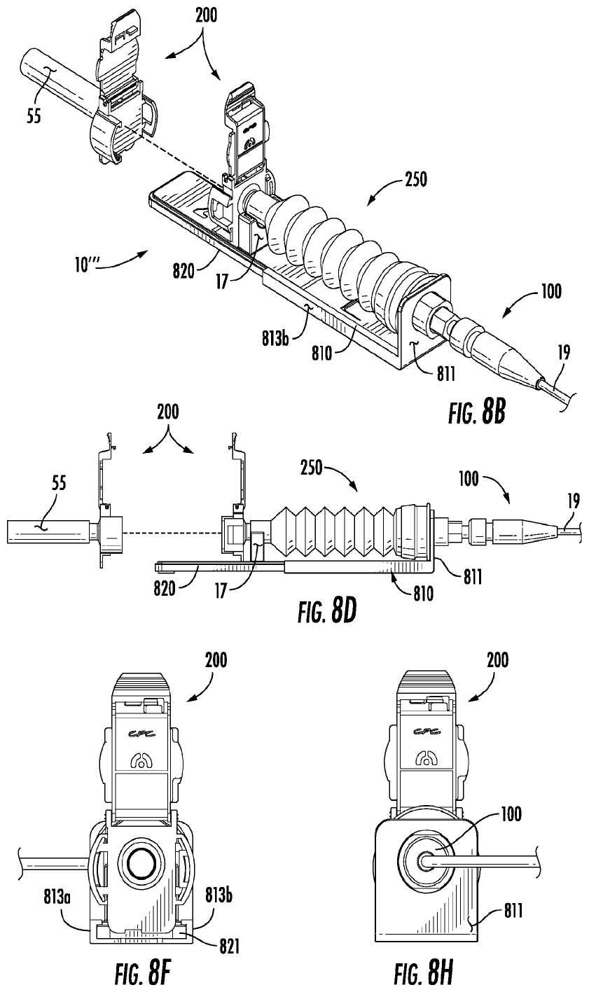

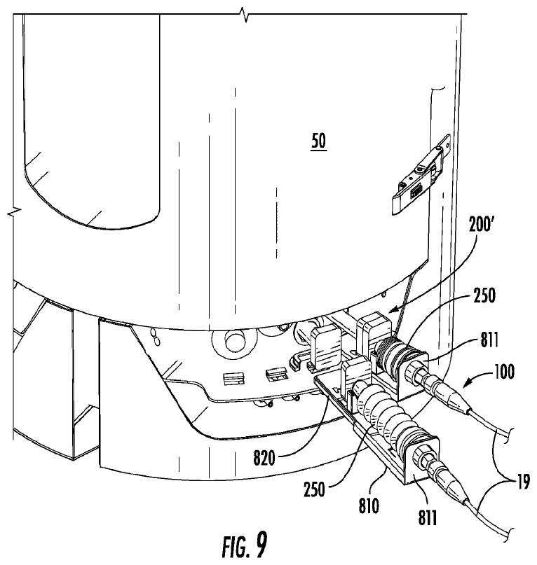

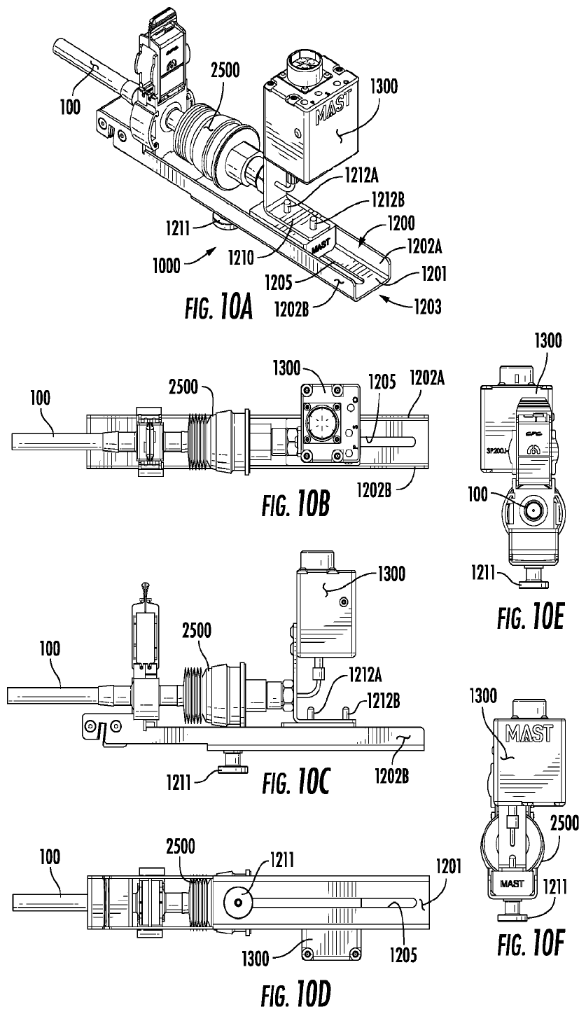

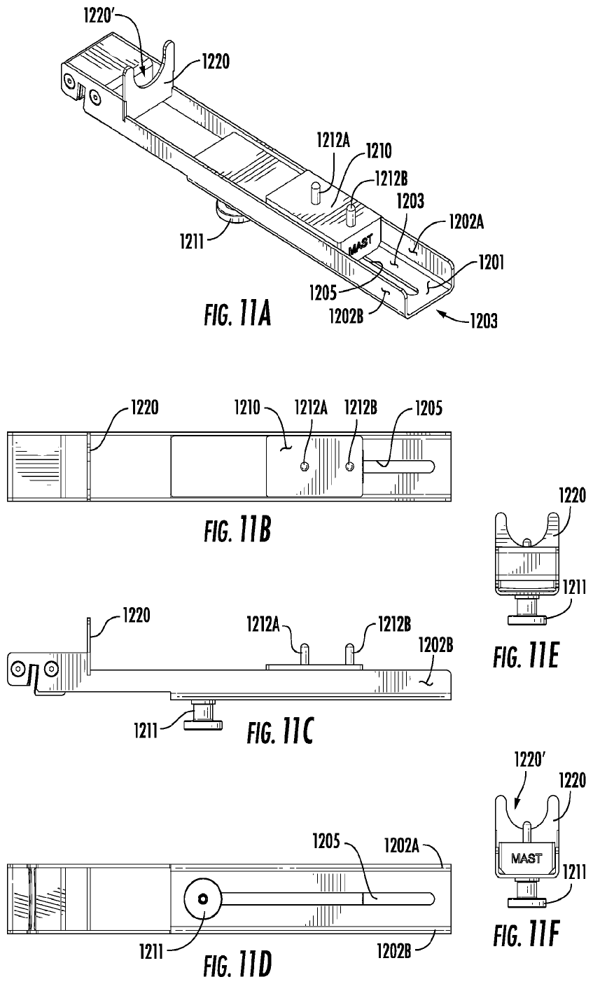

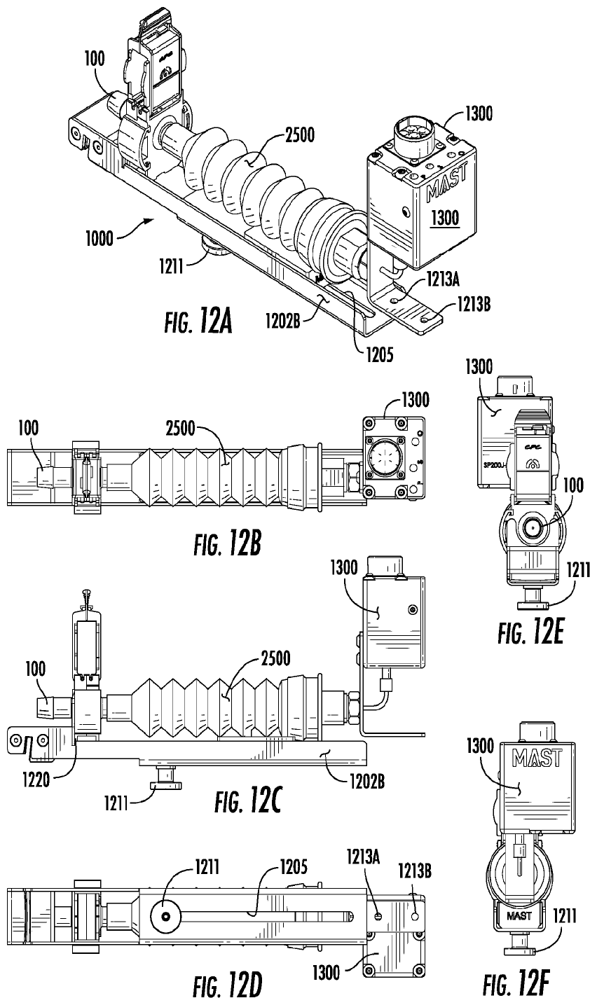

FIGS. 10-12 (e.g., FIGS. 10A, 10B, 10C, 10D, 10E, 10F, 11A, 11B, 11C, 11D, 11E, 11F, 12A, 12B, 12C, 12D, 12E, 12F, 12G and 12H) illustrates a fifth embodiment of a sensor holder 1000. This sensor holder 1000 is suitable for use in the MAST (Modular Automated Sampling for Technology), example, for aseptic bioreactor sampling. As shown in FIGS. 10A and 12H, the holder 1000 includes an elongated member 1200 that includes a channel 1203 defined by a base 1201 and opposite elongated side walls 1202A, 1202B, the base including a slot 1205. A movable block 1210 sits in in the channel and includes a member such as a peg or the like (not shown) that positions in the slot and is engageable by locking knob 1211 that may be actuated to lock the base in place at a desired location in the channel. In certain embodiments, the peg or the like and the locking knob 1211 may be a threaded engagement, whereby relative rotation of the locking knob and the peg or the like locks the block 1210 in position in the channel of the holder, or unlocks the block. In certain embodiments, the block 1210 may include two spaced fixation pins 1212A, 1212B extending upwardly from the block, positioned and configured to receive corresponding respective apertures 1213A, 1213B (FIG. 12A) in the auto-sampler body 1300. When the auto-sampler body 1300 is so positioned, and moves with the block. In certain embodiments, the holder 1000 also includes support member 1220 having a U-shaped or arcuate groove or cut-out 1220′ at a free end shaped and positioned to receive and support a region of the sensor as shown.

In some embodiments, the holder 1000 includes an expandable and collapsible bellows 2500 having an internal passageway configured to receive a portion of the probe 100. In some embodiments, the collapsible bellows 2500 is part of an assembly that includes a connector at one end and a probe adapter on the other end. This assembly may be wired to a support block as part of the holder assembly on the connector end and supported on the opposite probe end.

In certain embodiments, in its supporting position shown in FIGS. 12A-12G, the holder 1000 serves to support the probe 100 during sterilization, such as during autoclaving (FIG. 12A shows the deploying position with the auto-sampler body 1300 disengaged from the pins 1212A, 1212B). In this embodiment, the probe 100 is inserted through the bellows 2500 and the assembly sits on block 1210 and support member 1220. To deploy the probe 100, the holder 1000 is actuated so that block 1210 is translated linearly in slot 1205 of channel 1203 until the deploying position of FIG. 10A-10F and 12H is achieved In this position, the probe 100 is held in arcuate recess 1220′ as shown, is also supported by block 1210, and the bellows 2500 is in a compressed state as shown. The probe may be locked in this position by tightening locking knob 1211. The assembly then may be coupled to a bioreactor or other container by positioning it for such coupling.

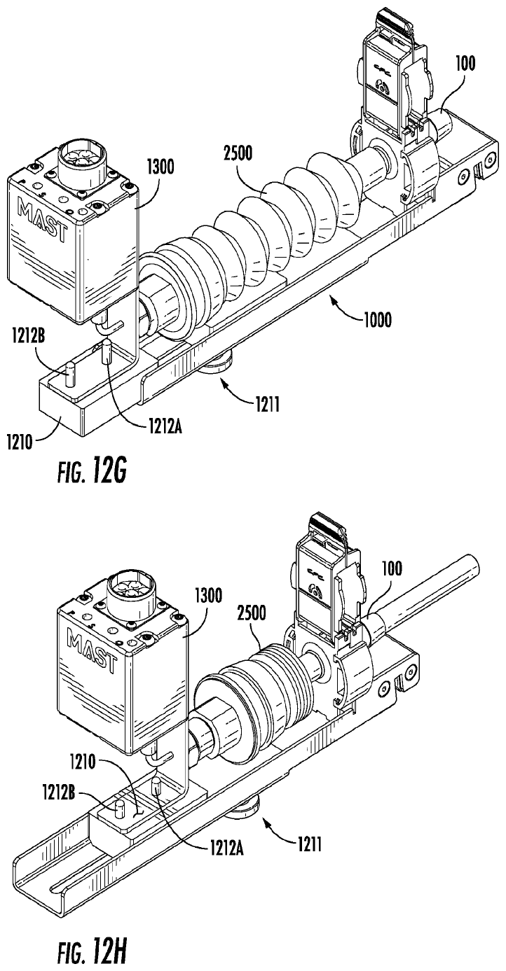

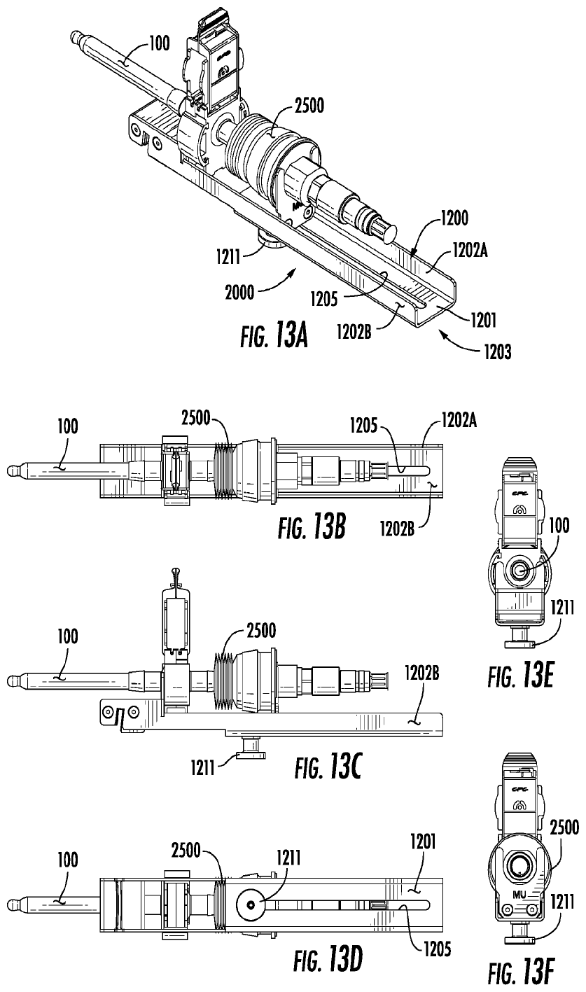

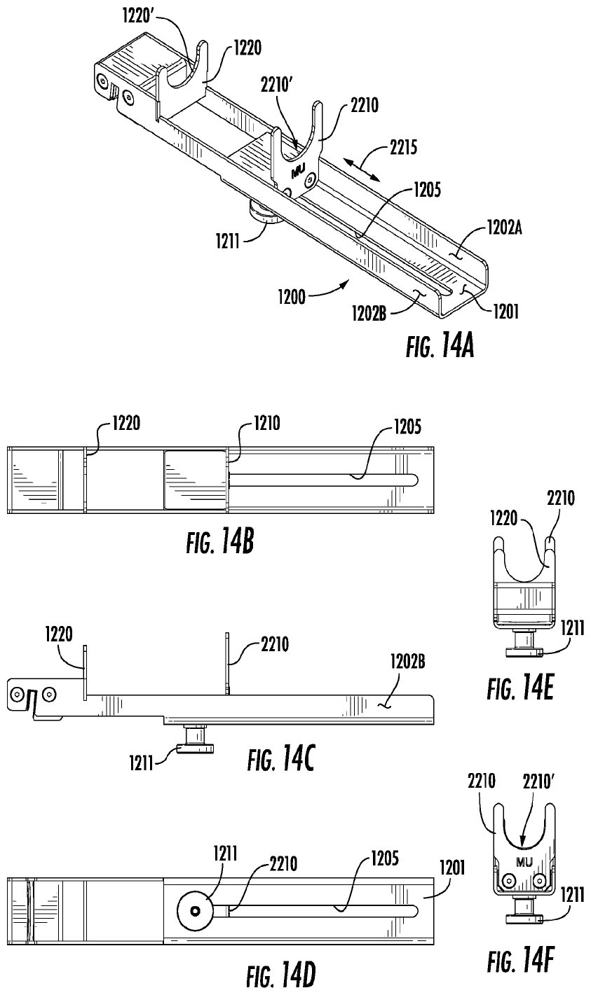

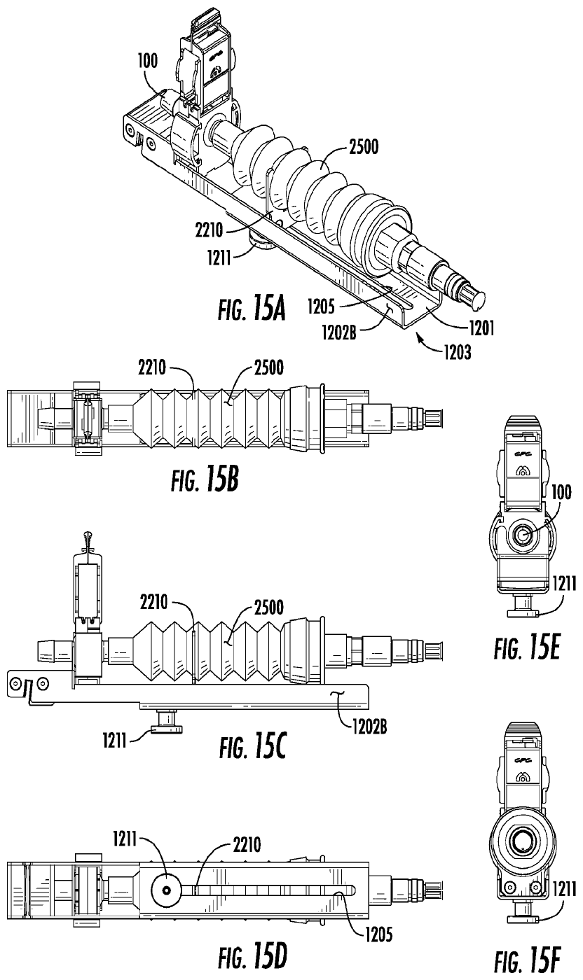

FIGS. 13-15 (e.g., FIGS. 13A, 13B, 13C, 13D, 13E, 13F, 14A, 14B, 14C, 14D, 14E, 14F, 15A, 15B, 15C, 15D, 15E and 15F) illustrates a sixth embodiment of a sensor holder 2000 particular suitable for a pH/DO/VCD/pCO₂ sensor. This embodiment is similar to the fifth embodiment of FIGS. 10-12, except that instead of moveable block 1210, there is movable U-shaped holder 2210. In certain embodiments, the U-shaped holder 2210 has a U-shaped or arcuate groove or cutout 2210′ at a free end having a similar shape to the groove or cutout 1220′, as best seen in FIGS. 14E and 14F, shaped and positioned to receive and support a region of the sensor as shown in FIG. 15A for example. Like the fifth embodiment, the holder 2000 includes an elongated member 1200 that includes a channel 1203 defined by a base 1201 and opposite elongated side walls 1202A, 1202B, the base including a slot 1205.

In certain embodiments, in its supporting position shown in FIGS. 15A-15F, the holder 2000 serves to support the probe 100 during sterilization, such as during autoclaving. In this embodiment, the probe 100 is inserted through the bellows 2500 and the assembly sits on U-shaped holder 2210 and support member 1220. To deploy the probe 100, the holder 2000 is actuated so that block U-shaped holder 2210 is translated linearly in slot 1205 of channel 1203 until the deploying position of FIG. 13A is achieved. In this position, the probe 100 is held in arcuate recesses 1220′ and 2210′ as shown, and the bellows 2500 is in a compressed state as shown. The probe may be locked in this position by tightening locking knob 1211. Thus, the block U-shaped holder 2210 is slidable back-and-forth in slot 1205 in the directions of arrow 2215 in FIG. 14A. The assembly then may be coupled to a bioreactor or other container by positioning it for such coupling, as in earlier embodiments.

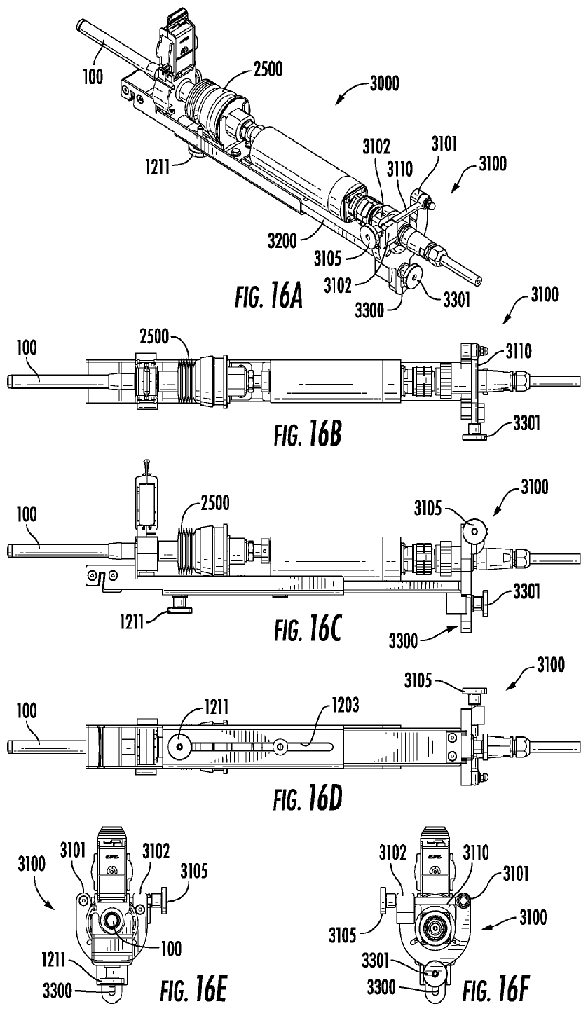

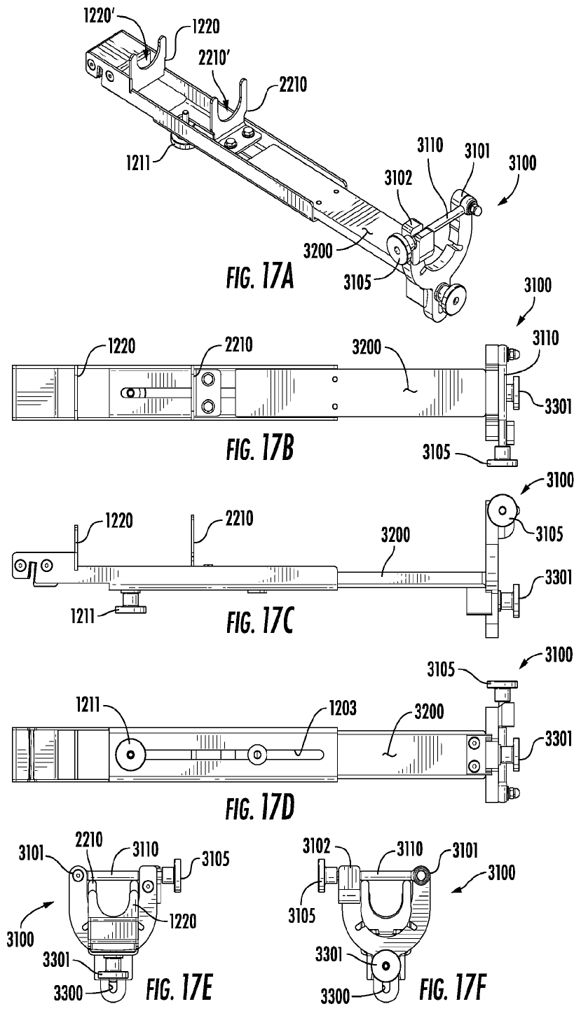

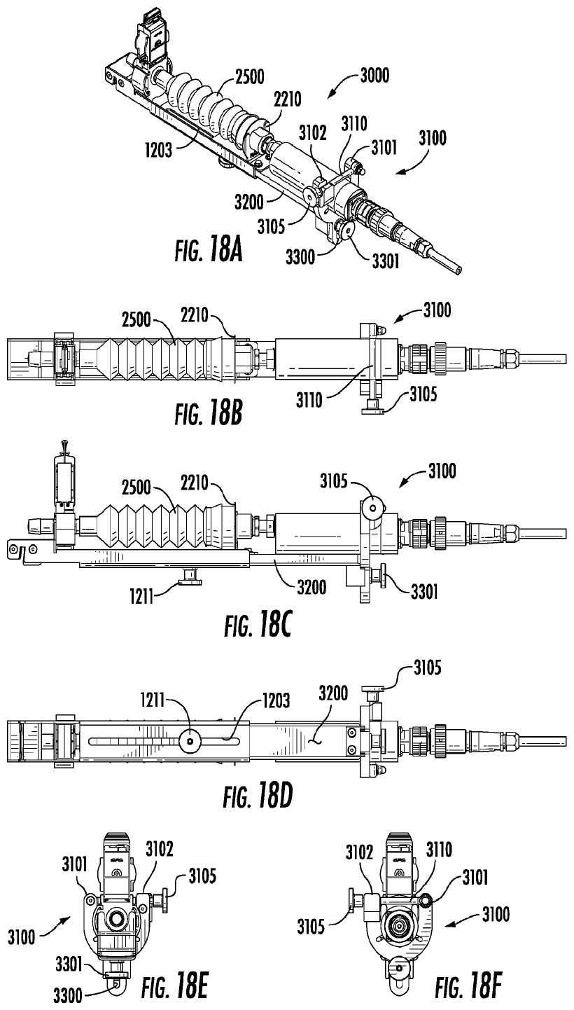

FIGS. 16-18 (e.g., FIGS. 16A, 16B, 16C, 16D, 16E, 16F, 17A, 17B, 17C, 17D, 17E, 17F, 18A, 18B, 18C, 18D, 18E and 18F) illustrates a seventh embodiment of a sensor holder 3000 particular suitable for Raman spectroscopy, such as ProCellics™ analyzer or sensor used as a tool for inline and real-time process analytics in bioprocessing. This embodiment is similar to the sixth embodiment of FIGS. 13-15, with the addition of a clamp assembly 3100 associated with a lineally translatable member 3200. In certain embodiments, the U-shaped holder 2210 has a U-shaped or arcuate groove or cutout 2210′ at a free end having a similar shape to the groove or cutout 1220′, as best seen in FIGS. 17E and 17F, shaped and positioned to receive and support a region of the sensor as shown in FIG. 18A for example. In certain embodiments, in its supporting position shown in FIGS. 18A-18F, the holder 3000 serves to support the probe 100 during sterilization, such as during autoclaving. In this embodiment, the probe 100 is inserted through the bellows 2500 and the assembly sits on U-shaped holder 2210 and support member 1220. It is also held in place by clamp assembly 3100, which in the embodiment shown is also generally U-shaped, and includes a locking bar 3110 that traverses the U-shaped opening when in the locked position shown. In certain embodiments, the locking bar 3100 is pivotable on free end 3101 of the U-shaped clamp assembly 3100, and is insertable into an open slot in the other free end 3102 of the clamp assembly 3100 and can be locked in place by locking knob 3105. In certain embodiments, the vertical position of the clamp assembly 3100 may be modified by sliding it vertically in slot 3300 and locking it in the desired position with locking knob 3301. To deploy the probe 100, the holder 3000 is actuated so that block U-shaped holder 2210 is translated linearly in channel 1203 until the deploying position of FIG. 16A is achieved. In this position, the probe 100 is held in arcuate recesses 1220′ and 2210′ and by clamp assembly 3100 as shown, and the bellows 2500 is in a compressed state as shown. The probe may be locked in this position by tightening locking knob 1211. The assembly then may be coupled to a bioreactor or other container by positioning it for such coupling.

The sensor 100 may be connected to a port of a container 50 via a conduit 55 or the like with a suitable coupling device 200, such as an AseptiQuik® G connector commercially available from Colder Products Company as with the previous embodiments.

What is claimed is:

1. A probe holder for positioning and holding a probe for engagement with a container, the probe having a longitudinal axis extendable in a longitudinal direction between a and being compressed position and an extended position, the holder comprising a first translatable member and a second translatable member, the first and second translatable members being translatable with respect to each other and configured to support said probe in said extended position and engage said probe in said compressed position.

2. The probe holder of claim 1, wherein said first and second translatable members are linearly translatable in the direction of said longitudinal axis.

3. The probe holder of claim 1, wherein said first and second translatable members are pivotably translatable about a pivot axis orthogonal to said longitudinal axis.

4. The probe holder of claim 1, further comprising an expandable and collapsible bellows having a passageway configured to receive said probe.

5. The probe holder of claim 4, wherein said first translatable member protects said expandable and collapsible bellows when said expandable and collapsible bellows is in a collapsed state.

6. The probe holder of claim 1, further comprising an aseptic connector.

7. The probe holder of claim 1, wherein the first translatable member slides in a slot or groove of the second translatable member.

8. The probe holder of claim 1, wherein said first and second translatable members are supported on a base.

9. The probe holder of claim 8, further comprising opposite spaced elongated side walls defining with said base a channel.

10. A probe holder for positioning and holding a probe for engagement with a container, the probe having a longitudinal axis and being extendable in a longitudinal direction between a compressed position and an extended position, the holder comprising a slotted base, a translatable member and a support member, the translatable member being translatable in said slotted base with respect to said support member.

11. The probe holder of claim 10, wherein said translatable member comprises a movable block that is lockable in said base.

12. The probe holder of claim 10, wherein said translatable member is U-shaped.

13. The probe holder of claim 10, further comprising a clamp assembly having a U-shaped recess and a locking bar that is positionable to traverse said U-shaped recess.

14. The probe holder of claim 13, wherein said clamp assembly has a first free end and a second free end defining therebetween said U-shaped recess, and wherein said locking bar is pivotable on said first free end.

15. The probe holder of claim 10, wherein said slotted base has two opposite spaced elongated side walls defining a channel between them.
